SEQUENCE LISTING (4 sequences)

SEQ ID NO: 1 - protein, 4 aa, Artificial Sequence
  Amino acid sequence involved in the activity of Factor Xa
IEGR

SEQ ID NO: 2 - protein, 7 aa, Artificial Sequence
  Amino acid sequence involved in the activity of TEV protease
ENLYFQG

SEQ ID NO: 3 - protein, 6 aa, Artificial Sequence
  Amino acid sequence involved in the activity of Thrombin
LVPRGS

SEQ ID NO: 4 - protein, 4 aa, Artificial Sequence
  Amino acid sequence site to be replaced by destabilization sequence
VDGK

(19) United States
(12) Patent Application Publication
(10) Pub. No.: US 2010/0196951 A1
(43) Pub. Date: Aug. 5, 2010

(54) Antitoxin Destabilization Technology

(75) Inventors: Masayori Inouye, New Brunswick, NJ (US); Koichi Inoue, Otsu (JP)

Correspondence Address: PATENT DOCKET ADMINISTRATOR;LOWENSTEIN SANDLER PC, 65 LIVINGSTON AVENUE, ROSELAND, NJ, 07068, US

(73) Assignee: UNIVERSITY OF MEDICINE AND DENTISTRY OF NEW JERSEY, Somerset, NJ (US)

(21) Appl. No.: 12/668,841

(22) PCT Filed: Jul. 14, 2008
(86) PCT No.: PCT/US08/70001

§ 371 Date: Jan. 12, 2010

Related U.S. Application Data
(60) Provisional application No. 60/959,399, filed on Jul. 12, 2007.

(51) Int. Cl. C12P 21/00; C07K 1/14; C12N 15/74

(52) U.S. Cl. 435/68.1; 530/412; 435/320.1

(57) ABSTRACT

Disclosed are methods for purifying toxin proteins which avoid denaturation and renaturation procedures.

### CLAIM OF PRIORITY

This application claims priority to U.S. Provisional Application No. 60/959,399, filed Jul. 12, 2007, the disclosure of which is hereby incorporated by reference in its entirety.

### STATEMENTS REGARDING SEQUENCE LISTINGS

Submitted herewith, in written and computer readable form, are listings of the amino acid sequences set forth herein. The attached sequence listings do not go beyond the disclosure in the international application as filed. The information recorded in computer readable form is identical to the written sequence listings related thereto.

### BACKGROUND OF THE INVENTION

Bacteria are generally equipped with the so-called toxin-antitoxin (TA) or “suicide” gene systems, which are considered to play important roles in growth regulation, cell death and dormancy under stress conditions. Under normal growth conditions, a toxin forms a stable complex with its cognate antitoxin encoded from the same operon (TA “complexes”), thus the toxin is incapacitated for acting on its cellular target. However, under stress conditions, labile antitoxins are rapidly degraded with concomitant release of free toxins in the cytoplasm, which then exert their toxic effect on specific cellular targets.

The number of toxin or suicide genes present on the bacterial genomes widely varies; Escherichia coli typically contains six independent TA operons, each encoding a pair of an antitoxin and its cognate toxin, while Mycobacterium tuberculosis contains approximately forty such operons. All the pathogenic bacterial genomes sequenced to date indeed contain one or more TA operons except for bacteria that live obligatorily with host cells such as Chlamydia and Mycoplasm. Out of six TA operons in E. coli, three have been well characterized; ReIE is a ribosome-associating factor that stimulates ribosomal endo-ribonuclease activity, and MazF and ChpBK act as sequence-specific endo-ribonucleases, termed mRNA interferases (Mlase), It has been demonstrated that MazF, when induced, cleaves cellular mRNAs at ACA sequences thereby effectively inhibiting cellular protein synthesis and thus cell growth. MazF forms a stable complex with its antitoxin, MazE, and the X-ray structure of the MazF-MazE complex has been determined. Since the TA complexes are not toxic to the cells, they are well expressed in E. coli and are readily purified with a very high yield. Recently, the X-ray structures of the ReIE-ReIB and the YoeB-YefM complexes have also been determined, revealing how toxins and antitoxins interact in the TA complexes.

Most bacteria contain a number of toxin or “suicide”' genes in their genomes. Importantly, the toxins produced from these genes are neither intended to kill other bacteria in their habitats nor to kill animal cells in the process of infection. Instead, they are produced intracellularly and are toxic to themselves. Recent developments in this new field have provided many intriguing insights into the role of these toxins in bacterial physiology, persistence in multi-drug resistance, pathogenicity, biofilm formation and evolution. It is now evident that the study of these toxins has very important implications in infectious diseases and medical sciences. Since most of these toxins are co-transcribed with their cognate antitoxins in an operon (thus termed as toxin-antitoxin or TA operons), and they form a stable complex in the cell under normal growth conditions, the toxic effect of these toxins is not typically exerted (Bayles, 2003; Engelberg-Kulka et al., 2004; Hayes, 2003; Rice and Baytes, 2003). However, since the stability of antitoxins is much less than that of their cognate toxins, any stress causing cellular damage or growth inhibition affects the balance between toxin and antitoxin in the cell, leading to release of toxins in the cell. Although much debated, it is most reasonable to consider that these toxins encoded from the TA operons function in two different ways depending upon the nature of the stress. One is to regulate the growth rate by inhibiting a particular cellular function such as DNA replication and protein synthesis. Under extensive stress, at which the amount of toxins exceeds the antitoxins, cell growth may be completely arrested. This role of TA toxins in growth regulation is likely to be their primary function. However, their second role is suicidal, that is to kill their own host cells. Under certain conditions, TA toxins may function to eliminate cells that are highly damaged (for example, DNA damage or phage infection) to maintain a healthy population. The TA operons are also often found in plasmids, which play a role in killing the cells that have lost plasmids after ceil division; a phenomenon known as post-segregational killing. Therefore, TA toxins are primarily bacteriostatic, but not bactericidal (Gerdes et al., 2005) but under certain conditions, cells may reach a point of no return resulting in cell death (Amitai et al., 2004). Recently, Engelberg-Kulka proposed that MazF, an E. coli toxin, is not an executioner of cell death but is rather a mediator that activates downstream systems (Engelberg-Kulka et al., 2005).

Since over-expression of toxins is known to be highly toxic for cell growth, it is essential to co-express their antitoxins for toxin purification. Conventional protocols for purification of toxin proteins from their TA complexes use His-tag affinity column chromatography followed by denaturation and renaturation procedures to remove antitoxins; these procedures are quite tedious.

Therefore, the need exists for an efficient method of purifying toxin proteins that does not require such denaturation and renaturation procedures.

### SUMMARY OF THE INVENTION

It is an object of certain embodiments of the present invention to provide a method for the purification of toxin proteins from their TA complexes which avoids the denaturation and renaturation procedures of previous conventional methods.

In certain embodiments, the present invention is directed to a method for disassociating a toxin protein from an antitoxin protein in a toxin-antitoxin complex comprising inserting a destabilization sequence into the toxin-antitoxin complex; and exposing the toxin-antitoxin complex to conditions to stimulate the co-expression of toxin and anti-toxin, wherein the destabilization sequence causes the antitoxin to disassociate from the toxin during co-expression.

In other embodiments, the present invention is directed to a method for disassociating a toxin protein from an antitoxin protein in a toxin-antitoxin complex comprising inserting a destabilization sequence into the antitoxin protein of a toxin-antitoxin complex; and exposing the toxin-antitoxin complex to conditions to stimulate the co-expression of toxin and anti-toxin, wherein the destabilization sequence causes the antitoxin to disassociate from the toxin during co-expression.

In other embodiments, the present invention is directed to a method for purifying a toxin protein in a toxin-antitoxin complex comprising inserting a destabilization sequence into the antitoxin protein of a toxin-antitoxin complex; exposing the toxin-antitoxin complex to conditions to stimulate the co-expression of toxin and anti-toxin, wherein the destabilization sequence causes the antitoxin to disassociate from the toxin during co-expression; and contacting the disassociated antitoxin with a suitable protease, wherein the protease digests the disassociated antitoxin, thereby purifying the toxin.

In further embodiments, the present invention is directed to a method of destabilizing a toxin-antitoxin complex comprising inserting a destabilization sequence into the toxin-antitoxin complex.

In other embodiments, the present invention is directed to a toxin-antitoxin co-expression system comprising genetic material encoding a toxin-antitoxin complex, wherein the toxin-antitoxin complex comprises a Thrombin recognition sequence (SEQ ID NO. 3).

In preferred embodiments, the destabilization sequence is a sequence capable of being cleaved by Factor Xa, TEV protease or Thrombin. In particularly preferred embodiments, the destabilization sequence is a Thrombin recognition sequence according to SEQ ID NO. 3.

### BRIEF DESCRIPTION OF THE DRAWINGS

FIG. 1 depicts the introduction of the Thrombin recognition sequence into the MazE antitoxin.

FIG. 2 depicts the removal of the residual MazE antitoxin from the purified MazF sample by Thrombin treatment.

FIG. 3 depicts a schematic model of one embodiment of the present invention.

FIG. 4 depicts the endoribonuclease activity of purified MazF.

### DETAILED DESCRIPTION OF THE INVENTION

As discussed above, almost all bacteria including human pathogens contain toxins, which form a stable complex (TA complexes) with their cognate antitoxins in the cells. In this fashion, under normal growth conditions, toxins are unable to exert their toxic effects in the cells. Since overexpression of toxins is known to be highly toxic for cell growth, it is essential to co-express their antitoxins for toxin purification. The inventors of the present invention have developed a highly efficient and simple method for purification of toxin proteins avoiding the denaturation and renaturation procedures.

In certain embodiments, toxin proteins may be purified by introducing a new amino acid sequence (the “destabilization sequence”) into the antitoxin protein to destabilize the antitoxin within the TA complex in the cell. The destabilization sequence may be a known sequence which may be cleaved by a specific protease such as Factor Xa, TEV protease and Thrombin. Factor Xa cleaves at amino acid recognition sequence IEGR (SEQ ID NO. 1); TEV protease cleaves at amino acid recognition sequence ENLYFQG (SEQ ID NO. 2); and Thrombin cleaves at amino acid recognition sequence LVPRGS (SEQ ID NO. 3).

If the three-dimensional structures of antitoxin proteins are known, the destabilization sequence may be inserted, e.g., at a loop region. As a result of the introduction of the foreign peptide, the antitoxin will destabilize, thereby disassociating the antitoxin from the TA complex during co-expression of the toxin and antitoxin in the cell. Since many antitoxins are known to be digested by intrinsic proteases such as Lon and ClpAP, the dissociated antitoxins will be easily removed by such proteases in vivo. By this way, the toxin protein can be purified without removing the antitoxin.

In certain embodiments, if the residual antitoxin is co-eluted with the toxin using one-step His-tag chromatography, the eluted toxin sample may be treated with specific proteases which recognize specific sequences introduced into the antitoxins.

The method of the present invention can be widely applied for any TA complex systems from any bacteria allowing a large scale isolation of toxins. The resulting purified toxins may be used not only for the basic sciences, but also as therapeutic tools for the treatment of human cancer and other diseases, e.g., use as non-conventional antibiotics. They may also be used for various industrial purposes.

### EXAMPLE

The method of the present invention is exemplified using the MazE-MazF system as a model, as described below.

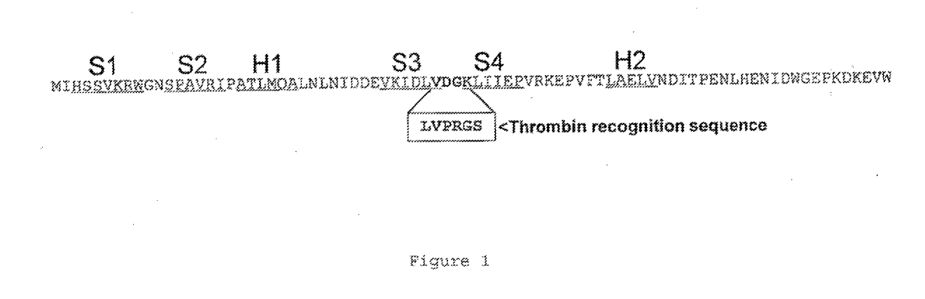

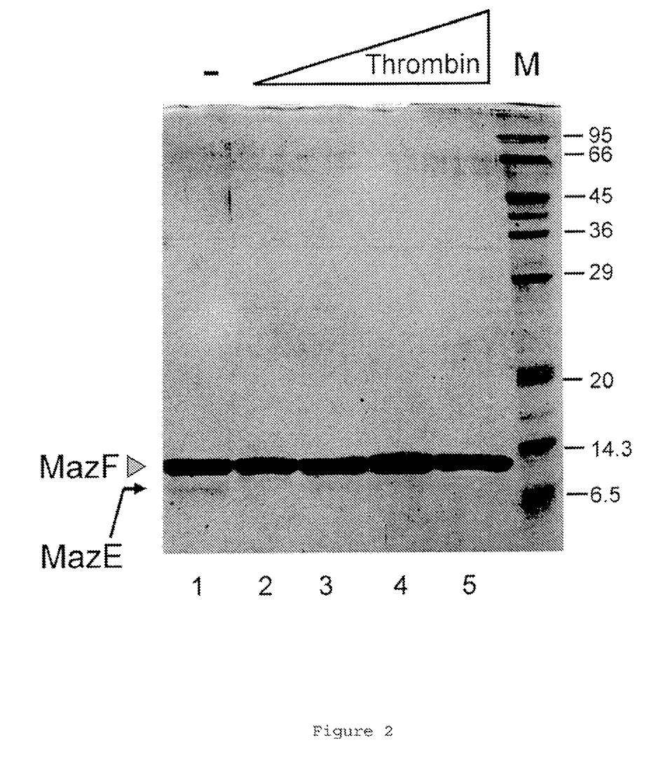

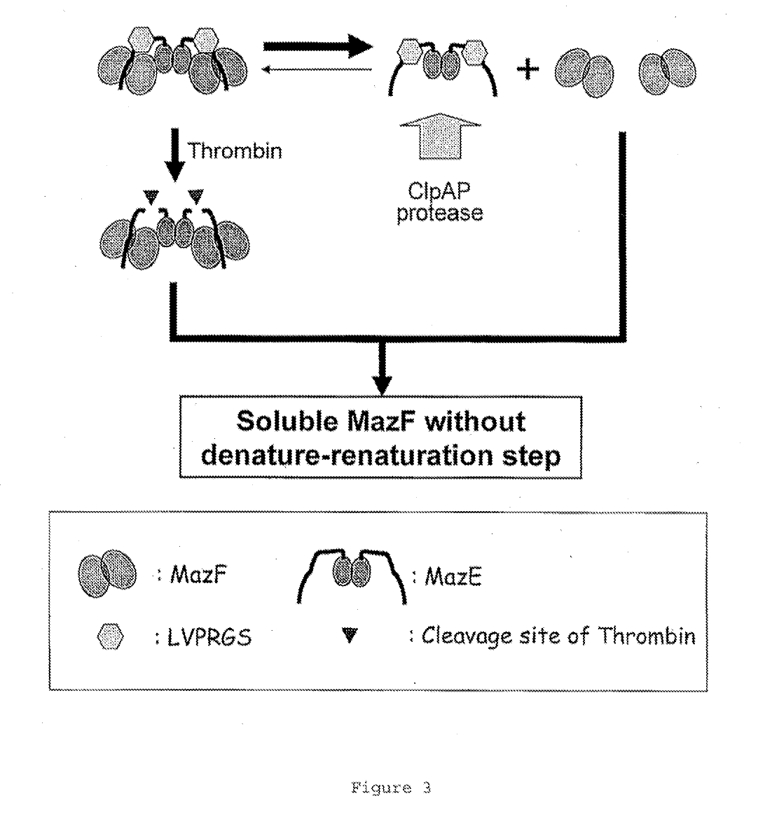

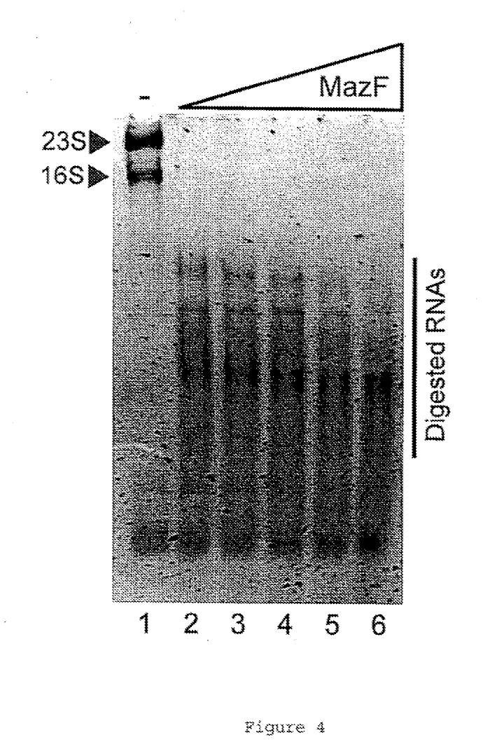

An IPTG-inducible mazE-mazF co-expression system was constructed in which the Thrombin-recognition sequence LVPRGS (SEQ ID NO. 3) was introduced into the loop region on MazE between beta strands S3 and S4 in place of the VDGK amino acid sequence located at positions 38 to 41 (SEQ ID NO: 4) (FIG. 1). The cells harboring this plasmid were incubated in the presence of IPTG to overexpress both MazE and MazF. The MazF sample purified with His-tag chromatography was incubated overnight at room temperature in the presence of none (lane 1), 0.01 units (lane 2), 0.02 units (lane 3), 0.04 units (lane 4), and 0.08 units (lane 5) of Biothinylated Thrombin (Novagen) (see FIG. 2). The samples were subjected to 15% SDS-polyacrylamide gel electrophoresis (“SDS-PAGE”), followed by Coornmasie-brillant Blue staining. MazE could not be detected on SDS-PAGE followed by the staining. It was hypothesized that the antitoxin, MazE, would be digested by endogenous proteases in vivo. His-tagged MazF protein was purified using His-tag chromatography, followed by dialyzing against 100 mM sodium phosphate (pH 7.6)-300 mM NaCl -20% glycerol-5 mM beta-mercaptoethanol. As shown in FIG. 2, highly pure MazF protein was obtained. Although a very faint MazE band was detected just below the MazF band on SDS-PAGE gel (lane 1), the residual MazE protein was successfully removed from the purified MazF sample by Thrombin treatment (lanes 2 to 5). The endoribonuclease activity of the purified MazF was confirmed by digesting total RNAs extracted form E. coli cells. Total RNAs extracted from E. coli cells with a hot phenol method were incubated for 10 minutes at 37° C. in the presence of none (lane 1), 0.375 ug (lane 2), 0.75 ug (lane 3), 1.5 ug (lane 4), 3 ug (lane 5) and 6 ug (lane 6) of purified MazF protein (see FIG. 4). The samples were subjected to 3.5% acrylamide gel electrophoresis, followed by ethidium bromide staining. A schematic model of this embodiment of the invention is shown in FIG. 3.

What is claimed:

1. A method for disassociating a toxin protein from an antitoxin protein in a toxin-antitoxin complex comprising:
a. inserting a destabilization sequence into the toxin-antitoxin complex; and
b. exposing the toxin-antitoxin complex to conditions to stimulate the co-expression of toxin and antitoxin, wherein the destabilization sequence causes the antitoxin to disassociate from the toxin during co-expression.

2. The method of claim 1, wherein the destabilization sequence is inserted into the antitoxin protein of the toxin-antitoxin complex.

3. The method of claim 1, wherein the destabilization sequence is a sequence capable of being cleaved by Factor Xa, TEV protease or Thrombin.

4. The method of claim 1, wherein the destabilization sequence is a Factor Xa recognition sequence according to SEQ ID NO. 1.

5. The method of claim 1, wherein the destabilization sequence is a TEV protease recognition sequence according to SEQ ID NO. 2.

6. The method of claim 1, wherein the destabilization sequence is a Thrombin recognition sequence according to SEQ ID NO. 3.

7. The method of claim 2, wherein the destabilization sequence is inserted into a loop region of the antitoxin protein.

8. A method for purifying a toxin protein in a toxin-antitoxin complex comprising:
a. inserting a destabilization sequence into the antitoxin protein of a toxin-antitoxin complex;
b. exposing the toxin-antitoxin complex to conditions to stimulate the co-expression of toxin and anti-toxin, wherein the destabilization sequence causes the antitoxin to disassociate from the toxin during co-expression; and
c. contacting the disassociated antitoxin with a suitable protease, wherein the protease digests the disassociated antitoxin, thereby purifying the toxin.

9. The method of claim 8, wherein the destabilization sequence is a sequence capable of being cleaved by Factor Xa, TEV or Thrombin.

10. The method of claim 8, wherein the destabilization sequence is a Factor Xa recognition sequence according to SEQ ID NO. 1.

11. The method of claim 8, wherein the destabilization sequence is a TEV protease recognition sequence according to SEQ ID NO. 2.

12. The method of claim 8, wherein the destabilization sequence is a Thrombin recognition sequence according to SEQ ID NO. 3.

13. The method of claim 8, wherein the destabilization sequence is inserted into a loop region of the antitoxin protein.

14. The method of claim 8, wherein the protease is contacted with the antitoxin in vivo.

15. The method of claim 8, wherein the protease is contacted with the antitoxin after co-elution with the toxin using one step His-tag chromatography.

16. A method of destabilizing a toxin-antitoxin complex comprising inserting a destabilization sequence into the toxin-antitoxin complex.

17. The method of claim 16, wherein the destabilization sequence is inserted into the antitoxin protein of the toxin-antitoxin complex.

18. The method of claim 16, wherein the destabilization sequence is a Factor Xa recognition sequence according to SEQ ID NO. 1.

19. The method of claim 16, wherein the destabilization sequence is a TEV protease recognition sequence according to SEQ ID NO. 2.

20. The method of claim 16, wherein the destabilization sequence is a Thrombin recognition sequence according to SEQ ID NO. 3.

21. A toxin-antitoxin co-expression system comprising genetic material encoding a toxin-antitoxin complex, wherein the toxin-antitoxin complex comprises SEQ ID NO. 3.

22. The toxin-antitoxin co-expression system of claim 21, wherein the toxin-antitoxin complex comprises MazF and MazE.

23. The toxin-antitoxin co-expression system of claim 22, wherein SEQ ID NO. 3 is located in the loop region of MazE between beta strands S3 and S4.

24. The toxin-antitoxin co-expression system of claim 21, wherein the genetic material is a plasmid.

25. A method for disassociating a toxin protein from an antitoxin protein in a toxin-antitoxin complex comprising:
a. inserting a destabilization sequence into the antitoxin protein of the toxin-antitoxin complex; and
b. exposing the toxin-antitoxin complex to conditions to stimulate the co-expression of toxin and anti-toxin, wherein the destabilization sequence causes the antitoxin to disassociate from the toxin during co-expression.
